(19) United States
(12) Patent Application Publication
(10) Pub. No.: US 2017/0112108 A1
(43) Pub. Date: Apr. 27, 2017

(54) GENETICALLY MODIFIED NON-HUMAN MAMMALS HAVING MODIFIED LIVER CELLS AND/OR TISSUE AND METHODS OF MAKING SAME

(71) Applicant: Hera Testing Laboratories, Inc., Lexington, KY (US)

(72) Inventors: John Stuart Crawford, Lexington, KY (US); Tseten Yeshi, Lexington, KY (US); Fallon Noto, Lexington, KY (US)

(21) Appl. No.: 15/299,685

(22) Filed: Oct. 21, 2016

Related U.S. Application Data
(60) Provisional application No. 62/245,049, filed on Oct. 22, 2015.

(51) Int. Cl. A01K 67/027; C12N 7/00; C12N 15/85; C12N 15/86

(52) U.S. Cl. CPC A01K 67/0278; C12N 15/86; C12N 7/00; C12N 15/8509; C12N 2810/6018; A01K 2217/052; A01K 2217/072; A01K 2217/15; A01K 2217/206; A01K 2217/30; A01K 2207/15; A01K 2267/0387; C12N 2830/008; C12N 2710/10045; C12N 2710/10043; C12N 2015/8527

(57) ABSTRACT

Disclosed are non-human mammals having a modified liver and methods of making such non-human mammals having a modified liver. The modified liver may be characterized by a non-germline, stable integration of a non-endogenous gene targeted to the liver of the non-human mammal. In certain aspects, the modified liver may have greater than at least 30% ablation of the endogenous hepatocyte population of the non-human mammal. In certain aspects, the non-human mammal may comprise at least 30% non-endogenous hepatocytes. The disclosed non-human mammals may be useful for pharmacology, drug absorption, distribution, metabolism, and excretion studies, collectively ADME and toxicology studies (ADME-tox), and/or drug screening.

### BACKGROUND OF THE INVENTION

The ability to humanize animal models would have many potential uses in drug development and research applications. In particular, humanization of animal livers would be particularly beneficial, as many hepatic enzymes are species specific and human enzymes for metabolism of drugs must be available to evaluate candidate pharmaceuticals. To date, however, efficient methods that successfully allow for humanization of rodents, in particular rats, have not been developed. Rather, existing methods that utilize germline mutagenesis require multiple genetic manipulations requiring laborious, complex, and lower efficiency in breeding and animal utility.

The key applications for humanized models are pharmacology, drug absorption, distribution, metabolism, and excretion, collectively ADME and toxicology studies (ADME-tox) as well as discovery studies such as hepatitis and other infectious disease research. Most studies have been traditionally run in outbred rodent models, such as the Sprague Dawley rat. However, maintaining transgenic colonies of outbred models is very difficult due to factors such as transgene instability. Collick et al. (1994) EMBO J. 13(23): 5745-5753. This has been a real hurdle in certain species such as the rat where despite years of effort, a humanized-liver rat model has not been successfully obtained.

### BRIEF SUMMARY

Disclosed are non-human mammals having a modified liver and methods of making such non-human mammals having a modified liver. The modified liver may be characterized by a non-germline, stable integration of a non-endogenous gene targeted to the liver of the non-human mammal. In certain aspects, the modified liver may have greater than at least 30% ablation of the endogenous hepatocyte population of the non-human mammal. In certain aspects, the non-human mammal may comprise at least 30% non-endogenous hepatocytes. The disclosed non-human mammals may be useful for pharmacology, drug absorption, distribution, metabolism, and excretion studies, collectively ADME and toxicology studies (ADME-tox), and/or drug screening.

### BRIEF DESCRIPTION OF THE DRAWINGS

FIG. 1. Overview of hybrid rAAV/PB system for in vivo liver-specific gene delivery: The PB transposase and vector carrying the TK “suicide gene” driven by a liver specific promoter may be delivered to immunodeficient rats via i.p. injection. Liver specific AAV capsid delivery followed by PB transposase mediated stable integration of TK gene into the genome. Using AAV transduction, the PB system integrates genes stably into the genome at high efficiency. TK suicide gene driven by liver specific promoters (LPS1, albumin or synthetic promoters) ablates endogenous rat hepatocytes following introduction of GCV. Following endogenous hepatocyte ablation human hepatocytes are implanted for engraftment.

FIG. 2. pAAV2 vector maps and annotated sequences for rAAV-PB (3402 bp) for in vivo delivery of piggyBac transposase to rat liver. The vector encodes the piggyBac transposase under the transcriptional control of Liver Specific Promoter 1 (LSP1). Lowercase bold: AAV2 ITRs; Lowercase italic underlined: LSP1 promoter; Uppercase bold: Gene Coding Sequence (PiggyBac); Lowercase underlined: SV40 polyA signal; Uppercase Italics: PB ITRs

FIG. 3. pAAV2 vectors maps and annotated sequences for rAAV-PB-GFP (2499 bp) for in vivo delivery of the GFP transgene to rat liver. The vector encodes for GFP under the transcriptional control of Liver Specific Promoter 1 (LSP1) for visual confirmation of effective delivery and expression of transgene in the liver. Lowercase bold: AAV2 ITRs; Lowercase italic underlined: LSP1 promoter; Uppercase bold: Gene Coding Sequence (GFP); Lowercase underlined: SV40 polyA signal; Uppercase Italics: PB ITRs

FIG. 4. pAAV2 vectors maps and annotated sequences for rAAV-PB-dTK (2376 bp) for in vivo delivery of the HSVtk transgene to rat liver. The vector encodes a truncated version of the HSVtk transgene (this may also be the full HSVtk gene) under the transcriptional control of Liver Specific Promoter 1 (LSP1). Lowercase bold: AAV2 ITRs; Lowercase italic underlined: LSP1 promoter; Uppercase bold: Gene Coding Sequence (TK); Lowercase underlined: SV40 polyA signal; Uppercase Italics: PB ITRs.

FIG. 5. Three males and three females (4week old adults) were injected (tail Vein) with 5e10 viral vector genomes (rAAV-PB-GFP+rAAV-PBo) per animal. PBo means piggyBac codon-optimized [Bradley et al Nucleic Acids Research, 2007, Vol. 35, No. 12] for mammalian expression or the super piggyBac transposase (hyperactive version) [Yusa et al PNAS-2011-1531-6] hyperactive piggyBac transposase. Livers were harvested and analyzed for GFP expression at: A) 7 days post injection and B) at 14 days post injection. No animal showed significant GFP expression in the liver.

FIG. 6. At 4 weeks old, 3 males and 3 females were injected (tail vein) with 2.5el1 viral vector genomes (rAAV-PB-GFP) per animal. Livers were harvested and analyzed for GFP expression at: A) day 7 post injection and B) day 14 post injection. No animal showed significant GFP expression in the liver.

FIG. 7. Neonatal rats at 3 days of age, 3 males and 3 females were injected intraperitoneally with 5e10 vector genomes each of rAAV-PB-GFP and rAAV-PBo. Their livers harvested and analyzed for GFP expression at A) 14 days post injection and B) 28 days post injection. C) At 28 days livers were further subjected to immunohistochemistry with anti-GFP antibody, which confirms in vivo gene delivery efficiency at about 20%.

FIG. 8. Three male and three female neonatal rats, were injected intraperitoneally with 5e10 vector genomes each (rAAV-PB-GFP+rAAV-PBo) on days 2, 4 and 6 after birth. Livers were collected for analysis 7 days post second injection and shows A) ˜70% of hepatocytes expressing GFP. B) Control animals injected with the same volume of PBS.

FIG. 9. Rag2 KO eliminates mature B cells. Splenocytes were collected from age matched homozygous Rag2 KO and wildtype animals and were analyzed by flow cytometry.

FIG. 10. Rag2 KO rats lack mature T cells. Thymocytes were collected from age-matched homozygous Rag2 KO and wildtype animals and were analyzed by flow cytometry. Rag2 KO animals were essentially athymic; only residual tissue remained weighing approximately 10% of the wildtype organ could be identified and collected for analysis.

FIG. 11. Il2rg KO Rats exhibit NK cell Deficiency. Splenocytes were collected from age matched homozygous Il2rg KO and wildtype animals, and analyzed by flow cytometry after staining for NK cell specific CD161a.

FIG. 12A and FIG. 12B. U87MG human glioblastoma tumor growth in Rag2 rats. FIG. 12A. All 6 Rag2 rats transplanted with U87MG grew tumors while none of the control wildtype rats (not shown here) did. Tumor diameter was measured twice a week after transplantation and their size-progression is shown here. FIG. 12B. Image of a visible U87MG tumor on a Rag2 rat.

### DETAILED DESCRIPTION

The practice of the present invention will employ, unless otherwise indicated, conventional techniques of immunology, molecular biology, microbiology, cell biology and recombinant DNA, which are within the skill of the art.

As used in the specification and claims, the singular form “a”, “an” and “the” include plural references unless the context clearly dictates otherwise. For example, the term “a cell” includes a plurality of cells, including mixtures thereof.

As used herein, the term “stable integration” means that the non-endogeonous gene is integrated into the genome of the host and can be expressed. The stably integrated gene remains in the genome of the host animal for the life of the animal. Descendants of the stably transfected cells can express the non-endogenous gene.

Germline mutagenesis of mice and rats has been demonstrated using traditional methods such as embryonic stem cells and pronuclear injection as well as more advanced techniques in genome editing with CRISPRs and transposons. However, the creation of highly complex germline models for humanization require multiple genetic manipulations which leads to laborious, complex and ultimately lower efficiency in breeding and animal utility. For example, one method for mouse cellular/tissue liver humanization is to generate a double knockout mouse targeting the Prkdc and Il2rg genes, resulting in an immunocompromised mouse in combination with an additional germline transgene insertion of the herpes simplex virus type 1 thymidine kinase (HSVtk) transgene, the TK-NOG mouse. Hasegawa et al. (2011) Biochem Biophys Res Comm. 405(3): 405-410. This mouse model can be used to ablate endogenous mouse hepatocytes after a brief exposure to a non-toxic dose of ganciclovir (GCV or FIAU), followed by transplantation of human liver cells that propagate and are stably maintained within the liver. However, transgenic mice harboring germline HSVtk transgene and TK-NOG mice have a lower breeding efficiency due to male sterility. In recent breeding studies with TK-NOG mice only about 50% of the entire breeding colony will transmit the transgene and can be used for humanization studies. Hasegawa et al. (2011) Biochemical and Biophysical Research Communications. 405(3): 405-410.

Immunocompromised models of the human disease, tyrosinemia type I (Fah−/−; Rag2−/−; Il2rg−/− triple knockout mice; referred to FRG mice) have been described in Azuma, H., et al., Nature biotechnology, 2007. 25(8): p. 903-910. The Fah (fumarylacetoeacetate hydrolase) mutation results in the accumulation of a toxic metabolites, maleylacetoacetate and fumarylacetoacetate, in the tyrosine metabolism pathway, which causes hepatotoxicity. Grompe, M., et al. Nature genetics, 1995. 10(4): p. 453-460. The mice require 2-[2-nitro-4-(trifluoromethyl)benzoyl] cyclohexane-1,3-dione (NTBC) supplementation for survival; without this agent hepatocyte cell death rapidly occurs and the mice die of acute liver failure.

Several limitations remain that limit the FRG mouse use for toxicity and screening. For example, host animals need to be maintained under constant and expensive NTBC treatment even after high human hepatocyte engraftment for long term >6 month studies to prevent hepatocellular carcinoma from residual mouse hepatocytes. Bissig, K.-D., et al. The Journal of clinical investigation, 2010. 120(3): p. 924. FAH loss also affects kidney function, and while NTBC mitigates the damage to both the liver and kidneys, human hepatocyte transplantation will not rescue defective kidney function. Thus, kidney damage is likely to affect long term ADME/Tox studies. Grompe, M., et al. Nature genetics, 1995. 10(4): p. 453-460. In addition, according to multiple toxicology industry thought leaders potential drug-drug interactions with NTBC may represent a serious concern for FRG models. In addition to these limitations, FRG options for creating rats with humanized livers are limited because FRG rats are not yet readily available from commercial sources.

Germline genetically humanized rodent models have been generated using described genome engineering methods (Devoy et al. (2012) Nature Reviews Genetics 13: 14-20). The same difficulty and laborious breeding issues also occur in these models. In order to fully utilize the genetically humanized models, expression of the human transgene should ideally be sufficient in multiple tissues, for example, the liver and kidney.

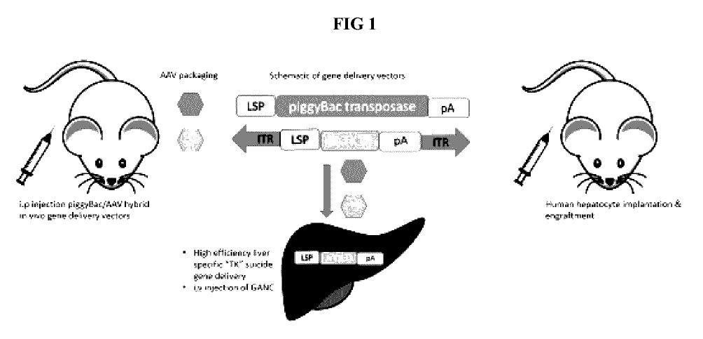

Recently a novel hybrid viral/non-viral recombinant-AAV/piggyBac transposon system has been utilized for the production of in vivo or non-germline transgenic mice expressing mouse transgenes in the liver. The system combines the highly efficient liver-targeting properties of rAAV serotypes with stable piggyBac-mediated transposition of the transgene into the hepatocyte genome, under the expression of a liver specific promoter [FIG 1]. The stable non-viral piggyBac mediated transgene maintains its stability as the endogenous liver cells expand [Cunningham et al. (2015) Hepatology 62: 417-429].

Disclosed herein are methods and animal models that effectively use the rAAV/piggyBac system for the introduction of non-germline transgenes for ablation of endogenous rodent hepatocytes for cellular or tissue humanization. Applicant has further found that the system may be efficiently used to introduce non-germline genetic humanization transgenes. Described herein are methods of using multiple tissue specific promoters including liver (e.g. albumin promoter, liver specific promoter LSP1) as well as novel synthetic tissue specific promoters designed for efficiency in delivery and transgene expression.

One challenge for increasing the efficiency of rAAV in vivo targeting is the size of the genetic cargo. For example, when traditional piggyBac inverted terminal repeats (ITRs) are used in the rAAV/piggyBac system a >20 fold increase in genomic integration of transgene is observed as measured by liver histological analysis (compared to delivery of transgene alone without piggyBac transposase). When the same system is used with smaller minimal piggyBac ITRs up to 103-fold increase in efficiency is observed. Disclosed are methods that address this problem via the use of novel, smaller, and more powerful synthetic tissue specific (e.g. liver, kidney) promoters.

In one aspect, a non-human mammal having a modified liver is disclosed. The modified liver is characterized by a non-germline, stable integration of a non-endogenous gene targeted to the liver of the non-human mammal. In one aspect, the non-endogenous gene may be selected from a gene having at least 85% identity, or at least 90% identity, or at least 95% identity, or at least 96% identity, or at least 97% identity, or at least 98% identity, or at least 99% identity, or 100% identity to a gene as set forth in the following table:

| Gene | | |
|---|---|---|
| (Alias) | Name | Gene ID |
| IFI44 | interferon induced protein | Human 10561 |
| | 44 | |
| CCDC85B | coiled-coiled domain | Human 11007 |
| | containing 85B | |
| URGCP | upregulator of cell | Human 55665 |
| (URG4) | proliferation | |
| ACY3 | aminoacylase 3 | Human 91703 |
| (HCBP1) | | |
| EXOC3L2 | exocyst complex | Human 90332 |
| | component 3 like 2 | |
| LAMTOR5 | late endosomal/lysosomal | Human 10542 |
| (HBXIP) | adaptor, MAPK and | |
| | MTOR activator 5 | |
| CD81 | CD81 | Human 975 |
| OCLN | occludin | Human |
| | | 100506658 |
| HB (X, X | X protein | Hepatitis B |
| protein) | | virus 944566 |
| HCVgp1 | polyprotein | Hepatitis C virus |
| | | 951475 |
| HCVgp2 | protein F | Hepatitis C virus |
| | | 951476 |
| PIM3 | Pim-3 proto-oncogene, | Human 415116 |
| | serine/threonine kinase | |
| MYC | v-myc avian | Human 4609 |
| | myelocytomatosis viral | |
| | oncogene homolog | |
| | [Homo sapiens | |
| E2F1 | E2F transcription factor 1 | Human 1869 |
| HRAS | HRas proto-oncogene, | Human 3265 |
| | GTPase | |
| TGFA | transforming growth | Human 7039 |
| | factor alpha | |
| PTEN | phosphatase and tensin | Human 5728 |
| | homolog | |
| PDGFRA | platelet derived growth | Human 5156 |
| | factor receptor alpha | |
| SREBF1 | sterol regulatory element | Human 6720 |
| | binding transcription | |
| | factor 1 | |
| PDGFC | platelet derived growth | Human 56034 |
| | factor C | |
| PLAUR | plasminogen activator, | Human 5329 |
| | urokinase receptor | |
| Cas9 | type II CRISPR RNA- | Streptococcus |
| | guided endonuclease | pyogenes |
| | Cas9 | WP_010922251 |
| Clo51 | | Clostridium |
| | | WP_008676092 |
| HFE | Hemochromatosis | Human 3077 |
| ATP7B | ATPase copper | Human 540 |
| | transporting beta | |
| SERPINA1 | serpin family A member 1 | Human 5265 |
| ALMS1 | ALMS1. centrosome and | Human 7840 |
| | basal body associated | |
| | protein | |

In one aspect, the modified liver may have greater than at least 30% ablation, or greater than at least 40%, or at least 50%, or at least 60%, or at least 70%, or at least 80%, or at least 90%, or at least 95% ablation of an endogenous hepatocyte population of said non-human mammal, wherein said percent ablation is determined by cell number.

In one aspect, the modified liver may comprise at least 30%, or at least 40%, or at least 50%, or at least 60%, or at least about 70%, or at least 80%, or at least 90%, or at least 95% of non-endogenous hepatocytes, wherein said percent non-endogenous hepatocytes is determined by cell number. In one aspect, the non-endogenous hepatocyte is selected from a human hepatocyte, a murine hepatocyte, a non-human primate hepatocyte, a canine hepatocyte, a feline hepatocyte or a combination thereof.

In one aspect, the non-human mammal may be selected from a mouse, a rat, a pig, or a rabbit. In a further aspect, the non-human mammal, may have an immunodeficient phenotype, such as, for example, a SCID phenotype.

In a further aspect, methods of using the disclosed modified animals are disclosed. The non-human mammals disclosed herein may be used for pharmacology, drug absorption, distribution, metabolism, and excretion, collectively ADME and toxicology studies (ADME-tox), or for drug screening. Such methods will be readily understood by one of ordinary skill in the art.

In a further aspect, methods of making the non-human mammal as described herein is disclosed. The method may comprise the steps of administering a first viral vector comprising a gene of interest; wherein said gene is operatively linked to a promoter. The promoter may be selected from a liver specific promoter, such as, for example, a Liver Specific Promoter 1 (LSP1) or albumin promoter. The first viral vector may be delivered to the non-human mammal via a method selected from hydrodynamic injection, intraperitoneal injection, or a combination thereof. The method may further comprise the step of administering a second viral vector comprising a transposase. In one aspect, the transposase may be selected from piggyBac, a Sleeping Beauty, To12, Mos1, Frog Prince (FP), and Buster. In a further aspect, the transposase sequence may be that described in U.S. Pat. No. 8,399,643, Ostertag et al., issued Mar. 19, 2013. The transposase may be operatively linked to a promoter. The promoter may be selected from a liver specific promoter, more preferably Liver Specific Promoter 1 (LSP1) or albumin promoter. The second viral vector may be delivered to the non-human mammal via a method selected from hydrodynamic injection, intraperitoneal injection, or a combination thereof. In another aspect, in vivo liver gene modification may also be achieved by direct hydrodynamic injection of DNA plasmids for genome engineering, for example, CRISPR/Cas9 and piggyBac transposon.

In one aspect, the viral vector comprises a recombinant adeno-associated viral vector (rAAV).

In one aspect, the gene of interest may be a suicide gene. Suicide genes include, but are not limited to, thymidine kinase (tk), for example, herpes simplex virus tk (HSVtk), hypoxanthine phosphoribosyltransferase (hprt), cytidine deaminase (codA), xanthine guanosine phosphoribosyltransferase (gpt), cytosine deaminase, carboxyl esterase, and combinations thereof. The suicide gene may be one that is activated by ganciclovir (GCV) injection over a period of time, ranging from about 1 day, about 1 to about 5 days, from about 1 to about 10 days, from about 1 to about 20 days, from about 1 to about 30 days, or from about 1 to about 50 days or more. The method may further comprise the step of implanting a human hepatocyte in a neonatal rat, such that the neonatal rat liver becomes a humanized liver containing human hepatocytes.

In one aspect, the gene of interest may comprise a transposon in which the suicide gene is flanked by a transposase target sequences on the 5′ and 3′ ends of the suicide gene. The transposase target sequences may comprise piggyBac inverted terminal repeats (ITRs).

The gene of interest and the transposase may be administered to said non-human mammal over a 2 to 5 day period, a 3 to 4 day period, or over a 3 day period.

In one aspect, the disclosed first and second vectors may be a single vector.

In a further aspect, the non-human mammal disclosed herein may retain normal fertility, in particular wherein said the non-human mammal is a male, more particularly wherein the non-human mammal is a male rat.

### EXAMPLES AND METHODS

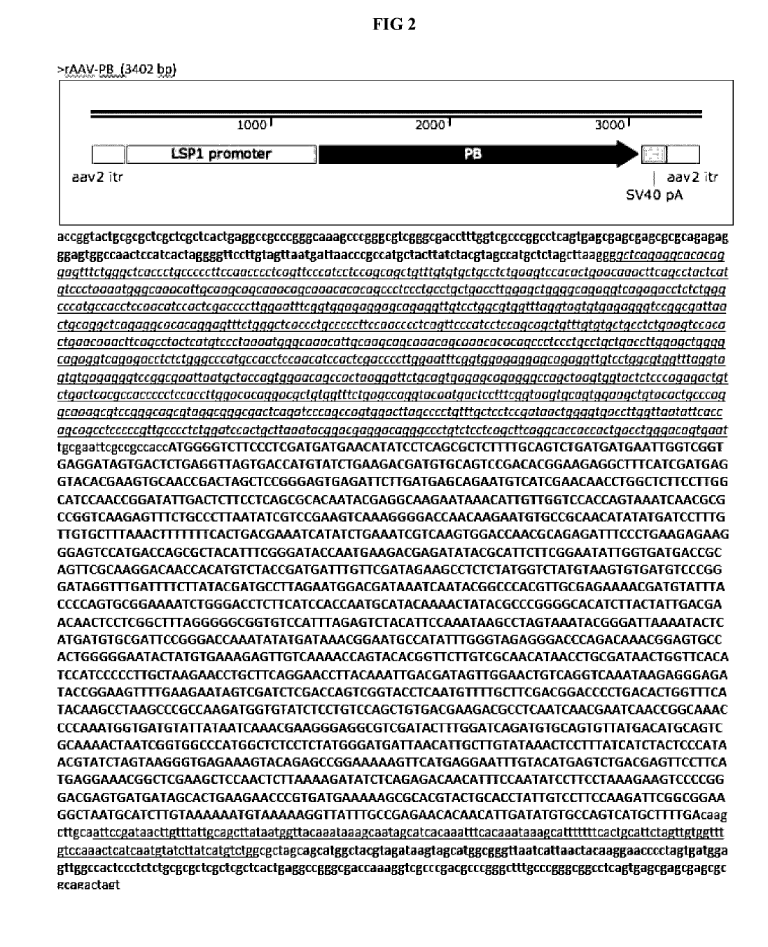

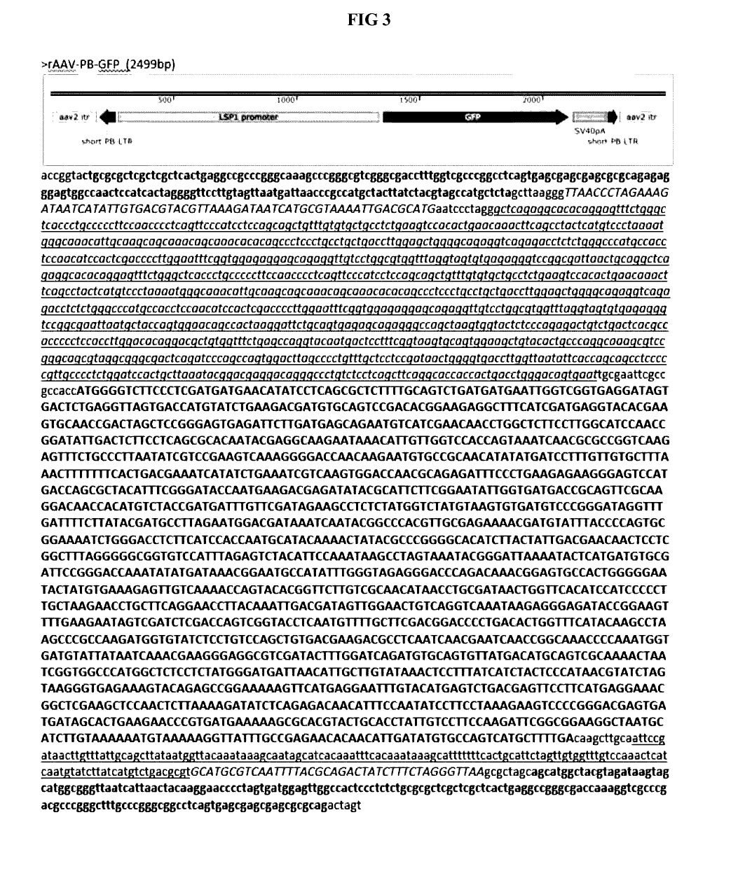

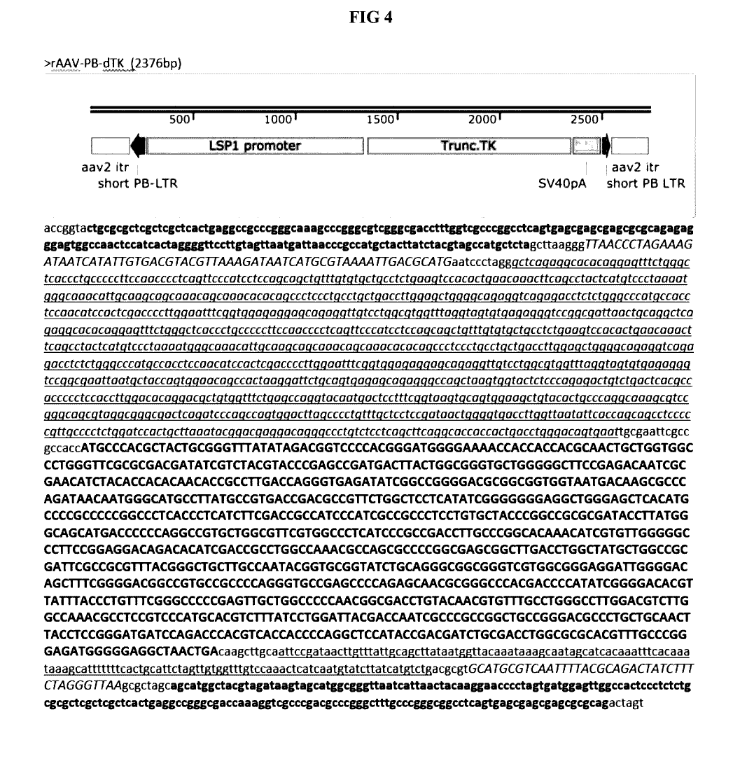

In vivo delivery of HSVtk as a piggyBac transposon by recombinant adeno-associated virus (rAAV) in the SCID Rat: An AAV vector containing a liver specific promoterl (LSP1) driving a truncated version of the HSVtk transgene was created [FIG. 4]. One of ordinary skill in the art will appreciate that the HSVtk gene may be used in its entirety. The construct also contains piggyBac inverted terminal repeats (ITRs) immediately internal to AAV ITRs that flank the HSVtk transgene. FIG. 4. A separate vector was created in which LSP1-piggyBac transposase is flanked by AAV inverted terminal repeats. FIG. 2. These two vectors were packaged into the capsid of an AAV serotype, using standard methods (Production and Titering of Recombinant Adeno-associated Viral Vectors. J. Vis. Exp. (57), e3348; Shin et al Methods Mol Biol. 2012 798 267-284; ABM Good Inc AAV Packaging Protocol) known to effectively target murine liver and introduced into the SCID rat by intraperitoneal injection for delivery to the liver. The suicide gene and the transposase could also be packaged into a single AAV vector and packaged into the capsid of a liver specific viral serotype. For proof of concept, Applicant utilized GFP or the HSVtk suicide gene and AAV2 vector, packaged into AAV serotype 8. FIG. 3, 4. The HSVtk gene could be replaced by any number of known suicide/negative selection genes including but not limited to hypoxanthine phosphoribosyltransferase (hprt), cytidine deaminase (codA), xanthine guanosine phosphoribosyltransferase (gpt), Cytosine Deaminase, Carboxyl Esterase, and inducible systems such as TET on/off system and Cumate system. Similarly, the AAV serotype may be other known variants, non-limiting examples including AAV1, AAV2, AAV7, AAV8, AAV9, AAVrh10d, or LK03 (LK03 as disclosed in U.S. Pat. No. 9,169,299). AAV serotypes are well known and are described in, for example, Lisowski et al, Adeno-associated virus serotypes for gene therapeutics, Current Opinion in Pharmacology, 2015.

Using methods similar to those described in the mouse (e.g., Cunningham et al.), 5e10 vector genomes was injected (tail vein) per animal into young adult (4weeks old) rats but this did not produce any significant delivery of GFP into rat livers. Specifically, three males and three females were injected with 5e10 viral vector genomes (rAAV-PB-GFP+rAAV-PBo) per animal. Livers were harvested and analyzed for GFP expression at 7 days and 14 days post injection. No animal showed significant GFP expression in the liver. FIG. 5.

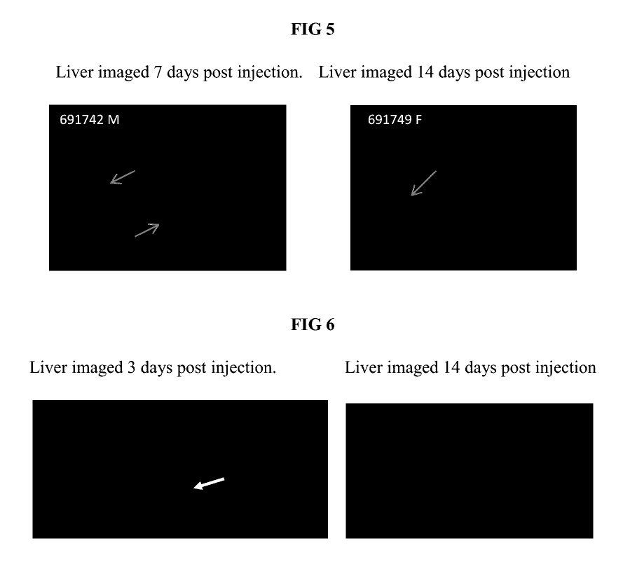

Increasing the amount of virus 5 fold to 2.5e11 vector genomes per animal vial tail vein did not improve in-vivo gene delivery. Since the rat is a significantly larger rodent than the mouse, it is possible the viral dose, shown to be effective for mice, is too low for rats. To test this, we increased the dose 5-fold to 2.5e11 vector genomes of rAAV-PB-GFP per rat via the tail vein. No improvement in GFP expression in the liver was observed over animals injected with 5e10 vector genomes. FIG. 6.

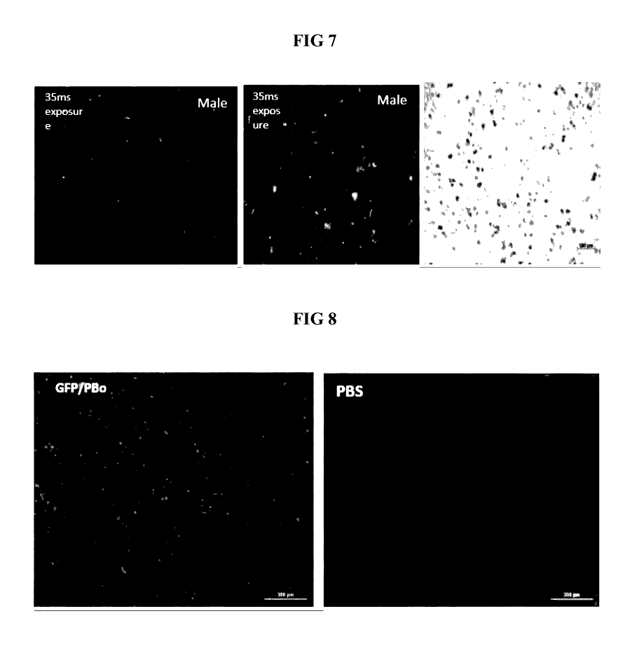

However, intraperitoneal injection (Amdrews K. IP Injections in the Mouse and Rat UBC Animal Care Guidelines; Turner et al Jaalas 2011 v50 n5 p600-613) into neonatal rats (1-4 days old) resulted in significant delivery of GFP to their livers albeit the efficiency was much lower than what's been shown in mice. 3 day old rats were injected intraperitoneally with 5e10 vector genomes each of rAAV-PB-GFP and rAAV-PBo. Analysis of their livers at 14 days post injection shows much better GFP expression in these livers (FIG. 7) compared to what we observed in adult rat livers. FIGS. 5 and 6. Further analysis of the liver at day 28 post injection shows that the GFP expression is stable and robust, and the efficiency of in vivo gene delivery by this method to neonatal rat livers is at about 20%. Recombinant rAAV episomal (or single stranded template) gene expression without stable integration by piggyBac clearance occurs within 4 weeks of age. FIG. 7. When using non-integrating rAAV by itself, gene expression in the liver is reduced to under 5% within 4 weeks [Cunningham et al. (2015) Hepatology 62: 417-429]. By utilizing the rAAV-PB-GFP and rAAV-PBo combination system, the applicants were able to prolong stable gene expression allowing for multiple compounding doses of gene delivery as well as ganciclovir to induce liver damage. (FIG. 7.)

20% targeted delivery is insufficient to induce desirable liver damage with ganciclovir (GCV). Three day old neonatal rats were injected with 5e10 vector genomes each of rAAV-PB-HSVtk and rAAV-PBo per animal. Due to the stable integration and expression of suicide gene HSVtk mediated by piggyBac transposon multiple injections of ganciclovir (GCV) can be administered over a prolonged period of time. 2 weeks later, each animal was injected with 50 mg/kg GCV (2 injections, 2 days apart). One week later blood serum was collected and analyzed for elevated ALT levels, which is indicative of liver damage. All animals showed normal ALT levels, suggesting there is no significant liver damage. ALT levels in the 200-300 range in desirable prior to transplantation of human hepatocytes.

Since 20% efficiency was not sufficient to induce desirable liver damage, and Because Applicant was limited by the volume that can be injected into the intraperitoneal cavity of neonatal rats at any one time, the amount of virus introduced was increased by injecting 5e10 vector genomes each on days 2, 4 and 6 after birth. Livers were collected for analysis 7 days post second injection. This method shows much improved efficiency (˜70%) of in vivo gene delivery to rat livers FIG. 8, which is comparable to the efficiencies previously shown in mice. It should also be noted from our earlier studies that GFP expression improves at day 14 compared to day 7.

Using the same multi-day viral introduction method as described in FIG. 8, GFP was delivered to the livers of both rats and mice. Both rAAV-PB-GFP and rAAV-PBo (labeled GFP/PBo in FIG. 7) with piggyBac transposase or rAAV-PB-GFP (labeled GFP in FIG. 7) without piggyBac transposase was used in the rat and just rAAV-PB-GFP without piggyBac transposase was used in the mouse. Livers were collected for analysis 28 days' post injection. The rat livers showed efficient stable introduction and expression of GFP in the rAAV-PB-GFP and rAAV-PBo with piggyBac transposase group. Unexpectedly, in the rAAV-PB-GFP without piggyBac transposase group, GFP expression was reduced to nearly zero or well below 5%, indicating that much fewer rAAV integration occurred without piggyBac in the rat than has been demonstrated in the mouse. The mouse rAAV-PB-GFP without piggyBac transposase showed nearly 20% GFP expression [FIG. 7].

Rat Background

Rag2 KO and Rag2; Il2rg double KO rats display SCID phenotype: Central to the ability to create rats with humanized livers was the generation of a SCID rat. Hera BioLabs used our proprietary site-specific DNA endonucleases (i.e., XTN™ TALENs and NextGEN™ CRISPR) to generate deletions within the Rag2 and Il2rg coding sequences in Sprague-Dawley rat. Homozygous colonies of rats carrying the deletions were generated for phenotypic analyses, which demonstrated that Rag2 and Il2rg knockout (KO) animals are indeed SCID in nature: The double knockout rats lack mature B cells, mature T cells and Natural Killer cells compared to a wild-type rat.

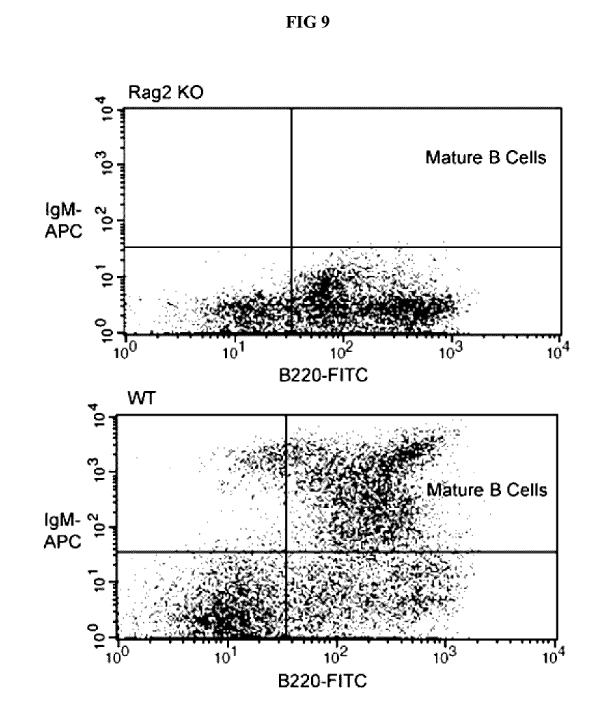

Splenocytes were analyzed for the markers B220 and IgM (B cell receptor) (see FIG. 7). Mutant animals had <0.2% double-positive splenocytes (i.e., mature B cells) while WT animals had 37.8% double-positive cells, which is the expected result with a Rag2 null phenotype since the B-cell receptor genes cannot undergo rearrangement. Isolated thymocytes were analyzed for CD4 and CD8 (see FIG. 8). The mutant thymocyte population contained only 5% double-positive cells, in contrast to the wildtype cells that were 89% double-positive. Thus, mutant animals fail to undergo normal T cell maturation. Il2rg KO rats exhibited deficits in NK cell differentiation as expected, resulting in >5-fold reduction in CD161a positive cells (a marker of NK cell identity; see FIG. 9). The Rag2 and Il2rg KO rats have intercrossed to generate a Rag2;Il2rg double KO rat, which maintains the immunodeficient phenotype.

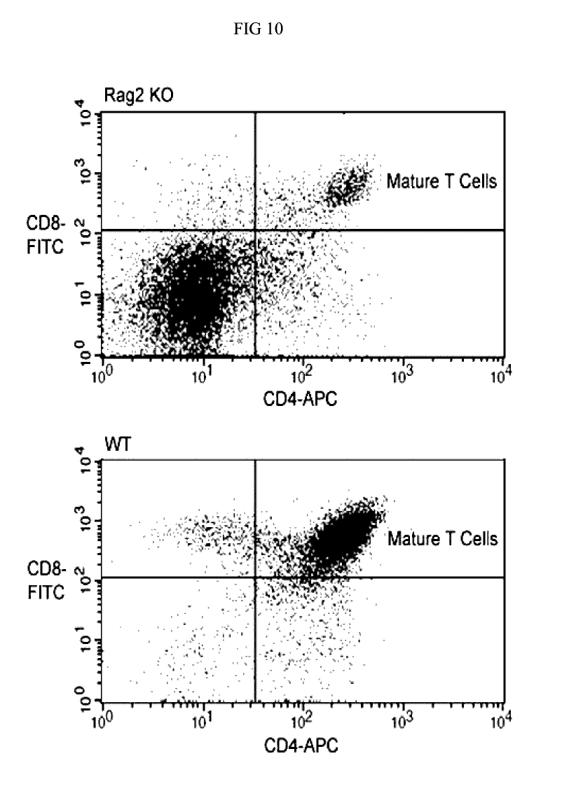

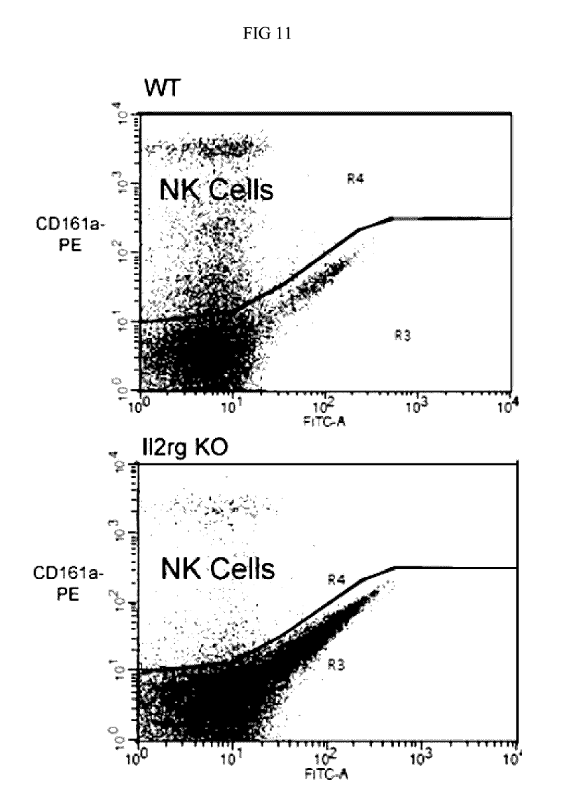

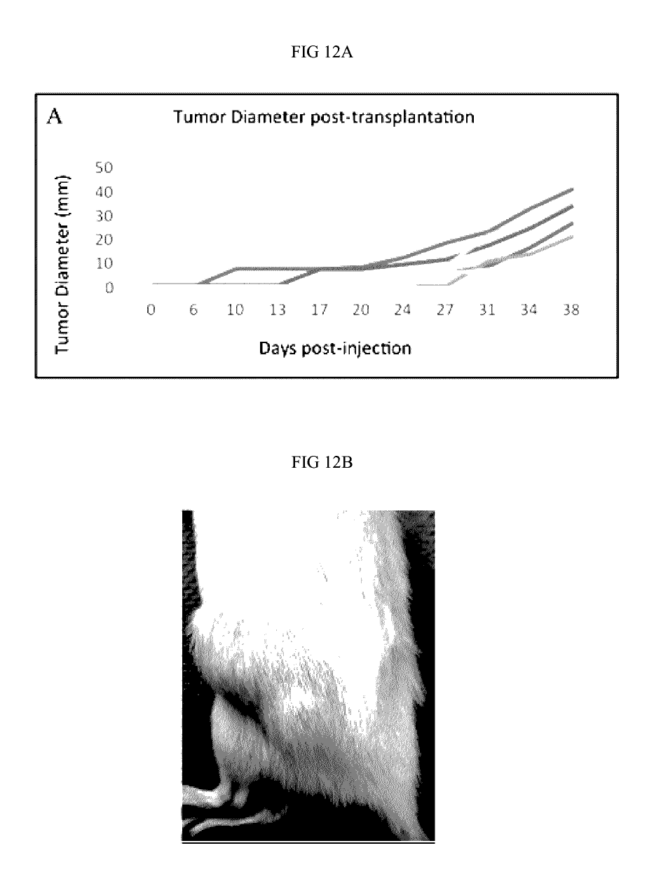

2. The Rag2 KO SCID rat does not reject subcutaneously transplanted U87MG human glioblastoma cell line. Applicant performed preliminary studies to demonstrate that the Rag2 mutation alone (eliminate B and T cell subsets of the immune system) allows the rat to accommodate human cells transplanted subcutaneously. The U87MG human glioblastoma cell line was implanted subcutaneously into three female and three male Rag2 knockout rats and three control wildtype rats. Rag2 rats had visible tumors as early as 10-days post implantation, and by day 27, all 6 Rag2 rats had visible and palpable tumors. None of the control wildtype rats grew tumors (FIG. 10).

Ablating Endogenous Rat Liver

The Herpes simplex virus typel thymidine kinase (TK) metabolizes the inert substrate ganciclovir (GCV) or FIAU into a toxic metabolite that induces cell death. Administration of GCV (0.5 to 50 mg/kg, IP) twice on alternate days is sufficient to kill heaptocytes that express HSVtk.

Description of Method for Transplanting Human Hepatocytes

Three animals each are transplanted via intrasplenic injection with 1, 2, or 5 million human primary hepatocytes, which may be conditionally immortalized hepatocytes and/or stem cell derived hepatocytes such as Corning® hepatocells or iCell® hepatocytes (hepatocells are an immortalized-cell-derived hepatocyte, iCell® hepatocytes are induced-pluripotent-stem-cell-derived hepatocytes). To increase the efficiency of engraftment, Applicant has suppressed the residual immune system of these SCID rats by treating them weekly with anti-asialo GM1 antibody from the day before transplantation and FK506 every 3 days after transplantation.

Methods of determining % Ablation AND % Humanization

Human albumin in serum of all animals every 2 weeks post transplant can be measured. Correlation of human albumin in the serum with percent repopulation can be demonstrated using the method described by Bissig et al. [14]. Animals can be monitored over time in this manner. Three animals were euthanized for histological analysis of liver tissue at the following time points: 1-week post-transplant to assess initial cell engraftment; 4-weeks; 8-weeks and 12-weeks post-transplant. Histological analysis of liver tissue sections can be examined for human albumin, CYP3A4, CYP2C18, CYP2C9, and al anti-trypsin, positive cells to assess repopulation efficiencies.

Following successful humanization of the liver, it can be confirmed to be functional and capable of metabolizing compounds in a human-specific manner. To validate human-specific function in vivo, humanized rats can be treated with FIAU and Bosetan, which are known to cause human-specific hepatotoxicity.

FIAU, an experimental Hepatitis B drug, cleared all preclinical toxicology studies in mice, rats, dogs and primates. It was subsequently administered to 15 clinical trial participants but seven participants developed acute liver failure: five died and two required a liver transplant [18, 2]. Age-matched control and humanized-liver rats were treated with FIAU at 2.5, 25, 100 or 400 mg/kg/day by oral gavage for 4-14 days. Then liver tissue was harvested for histology and immunohistochemistry analyses for signs of hepatotoxicity. Blood samples were collected for serum analyses to look for elevated lactate and ALT levels, which indicate liver damage.

Bosentan, an approved drug for arterial hypertension causes hepatotoxicity in a human-specific manner [19]. However, bosentan-treated rats do not develop hepatotoxicity [20]. Age matched control and humanized rats were treated with bosentan at 40, 80 or 160 mg/kg/day via oral gavage for 28 days. Animal health and weight was observed 3 times a week over the course of the experiment. Liver tissue was harvested on day 29 for histology and immunohistochemistry analyses for signs of hepatotoxicity. Blood was also collected from the animals for serum analyses via ELISA assays to look for elevated serum ALT, alkaline phosphatase and GGT levels, which are indicative of liver damage.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A non-human mammal having a modified liver, wherein said modified liver is characterized by a non-germline, stable integration of a non-endogenous gene targeted to the liver of the non-human mammal.

2. The non-human mammal of claim 1, wherein said non-endogenous gene is selected from a gene having at least 85% identity to a gene selected from IFI44, CCDC85B, URGCP (URG4), ACY3 (HCBP1), EXOC3L2, LAMTOR5 (HBXIP), CD81, OCLN, HBx (X, X protein), HCVgp1, HCVgp2, PIM3, MYC, E2F1, HRAS, TGFA, PTEN, PDGFRA, SREBF1, PDGFC, PLAUR, Cas9, Clo51, HFE, ATP7B, SERPINA1, ALMS1IFI44, and combinations thereof.

3. The non-human mammal of claim 1, wherein said modified liver has greater than at least 30% ablation of an endogenous hepatocyte population of said non-human mammal, wherein said percent ablation is determined by cell number.

4. The non-human mammal of claim 1, wherein said modified liver comprises at least 30% of non-endogenous hepatocytes, wherein said percent non-endogenous hepatocytes is determined by cell number.

5. The non-human mammal of claim 1, wherein said non-human mammal is selected from a mouse, a rat, a pig, or a rabbit.

6. The non-human mammal of claim 1, wherein said non-endogenous hepatocyte is selected from a human hepatocyte, a murine hepatocyte, a non-human primate hepatocyte, a canine hepatocyte, a feline hepatocyte or a combination thereof.

7. The non-human mammal of claim 1, wherein said non-human mammal has an immunodeficient phenotype.

8. A method of conducting a study selected from pharmacology, drug absorption, distribution, metabolism, and excretion, drug screening, or combinations thereof, comprising contacting a drug of interest with the non-human mammal of claim 1, or a a cell of a non-human mammal of claim 1.

9. A method of making the non-human mammal of claim 1, comprising the steps of
a. administering a first viral vector comprising a non-endogenous gene of interest;
wherein said gene is operatively linked to a promoter; and
b. administering a second viral vector comprising a transposase.

10. The method of claim 9, wherein said promoter is a liver specific promoter.

11. The method of claim 9, wherein said promoter is selected from a Liver Specific Promoter 1 (LSP1) or an albumin promoter.

12. The method of claim 9, wherein said transposase of step b is selected from piggyBac, a Sleeping Beauty, To12, Mos1, Frog Prince (FP), and Buster.

13. The method of claim 9, wherein said transposase of step b is operatively linked to a promoter.

14. The method of claim 13, wherein said promoter is selected from a liver specific promoter.

15. The method of claim 13, wherein said promoter is Liver Specific Promoter 1 (LSP1) or albumin promoter.

16. The method of claim 9, wherein said first and/or second viral vector is delivered to said non-human mammal via a method selected from hydrodynamic injection, intraperitoneal injection, or a combination thereof.

17. The method of claim 9, wherein said viral vector comprises a recombinant adeno-associated viral vector (rAAV).

18. The method of claim 9, wherein said non-endogenous gene of interest has at least 85% identity to a gene selected from IF144, CCDC85B, URGCP (URG4), ACY3 (HCBP1), EXOC3L2, LAMTOR5 (HBXIP), CD81, OCLN, HBx (X, X protein), HCVgp1, HCVgp2, PIM3, MYC, E2F1, HRAS, TGFA, PTEN, PDGFRA, SREBF1, PDGFC, PLAUR, Cas9, Clo51, HFE, ATP7B, SERPINA1, ALMS1IFI44, and combinations thereof.

19. The method of claim 9, wherein said non-endogenous gene of interest is a suicide gene, preferably wherein said suicide gene or is selected from thymidine kinase (tk), for example, herpes simplex virus tk (HSVtk), hypoxanthine phosphoribosyltransferase (hprt), cytidine deaminase (codA), xanthine guanosine phosphoribosyltransferase (gpt), cytosine deaminase, carboxyl esterase, and combinations thereof, more preferably wherein said suicide gene is activated by ganciclovir (GCV) injection over a period of time, ranging from 1 day, 1-5 days, 1-10 days, 1-20 days, 1-30 days, 1-50 days or more, preferably further comprising the step of implanting a human hepatocyte in said neonatal rat.

20. The method of claim 19, wherein said suicide gene is flanked by transposase target sequences on the 5′ and 3′ ends of said suicide gene.

21. The method of claim 20, wherein said transposase target sequences comprise piggyBac inverted terminal repeats (ITRs).

22. The method of claim 9, wherein said non-endogenous gene of interest and transposase are administered to said non-human mammal over a 2 to 5 day period.

23. The method of claim 9, wherein said first and second vector are a single vector.

24. The non-human mammal of claim 1, wherein said non-human mammal retains normal fertility, preferably wherein said non-human mammal is a male.


SEQUENCE LISTING (5 sequences)

SEQ ID NO: 1 - DNA, 1061 bp, Artificial Sequence
  LPS Promoter
gctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 2 - DNA, 193 bp, Rattus norvegicus
agcttcagatggcaaacatacgcaagggatttagttaaacaacttttttttttctttttggcaaggatggtatgattttgtaatggggtaggaaccaatgaactgaaaggttagtgtggttaatgatctacagttattggttagagaagtatattagagcgagtttctctgcacacagaccacctttcctgtc

SEQ ID NO: 3 - DNA, 3402 bp, Artificial Sequence
  rAAV-PB construct
accggtactgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcttaaggggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattgcgaattcgccgccaccatggggtcttccctcgatgatgaacatatcctcagcgctcttttgcagtctgatgatgaattggtcggtgaggatagtgactctgaggttagtgaccatgtatctgaagacgatgtgcagtccgacacggaagaggctttcatcgatgaggtacacgaagtgcaaccgactagctccgggagtgagattcttgatgagcagaatgtcatcgaacaacctggctcttccttggcatccaaccggatattgactcttcctcagcgcacaatacgaggcaagaataaacattgttggtccaccagtaaatcaacgcgccggtcaagagtttctgcccttaatatcgtccgaagtcaaaggggaccaacaagaatgtgccgcaacatatatgatcctttgttgtgctttaaactttttttcactgacgaaatcatatctgaaatcgtcaagtggaccaacgcagagatttccctgaagagaagggagtccatgaccagcgctacatttcgggataccaatgaagacgagatatacgcattcttcggaatattggtgatgaccgcagttcgcaaggacaaccacatgtctaccgatgatttgttcgatagaagcctctctatggtctatgtaagtgtgatgtcccgggataggtttgattttcttatacgatgccttagaatggacgataaatcaatacggcccacgttgcgagaaaacgatgtatttaccccagtgcggaaaatctgggacctcttcatccaccaatgcatacaaaactatacgcccggggcacatcttactattgacgaacaactcctcggctttagggggcggtgtccatttagagtctacattccaaataagcctagtaaatacgggattaaaatactcatgatgtgcgattccgggaccaaatatatgataaacggaatgccatatttgggtagagggacccagacaaacggagtgccactgggggaatactatgtgaaagagttgtcaaaaccagtacacggttcttgtcgcaacataacctgcgataactggttcacatccatcccccttgctaagaacctgcttcaggaaccttacaaattgacgatagttggaactgtcaggtcaaataagagggagataccggaagttttgaagaatagtcgatctcgaccagtcggtacctcaatgttttgcttcgacggacccctgacactggtttcatacaagcctaagcccgccaagatggtgtatctcctgtccagctgtgacgaagacgcctcaatcaacgaatcaaccggcaaaccccaaatggtgatgtattataatcaaacgaagggaggcgtcgatactttggatcagatgtgcagtgttatgacatgcagtcgcaaaactaatcggtggcccatggctctcctctatgggatgattaacattgcttgtataaactcctttatcatctactcccataacgtatctagtaagggtgagaaagtacagagccggaaaaagttcatgaggaatttgtacatgagtctgacgagttccttcatgaggaaacggctcgaagctccaactcttaaaagatatctcagagacaacatttccaatatccttcctaaagaagtccccgggacgagtgatgatagcactgaagaacccgtgatgaaaaagcgcacgtactgcacctattgtccttccaagattcggcggaaggctaatgcatcttgtaaaaaatgtaaaaaggttatttgccgagaacacaacattgatatgtgccagtcatgcttttgacaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctggcgctagcagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagactagt

SEQ ID NO: 4 - DNA, 3525 bp, Artificial Sequence
  rAAV-PB-GFP construct
accggtactgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcttaagggttaaccctagaaagataatcatattgtgacgtacgttaaagataatcatgcgtaaaattgacgcatgaatccctagggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattgcgaattcgccgccaccatggggtcttccctcgatgatgaacatatcctcagcgctcttttgcagtctgatgatgaattggtcggtgaggatagtgactctgaggttagtgaccatgtatctgaagacgatgtgcagtccgacacggaagaggctttcatcgatgaggtacacgaagtgcaaccgactagctccgggagtgagattcttgatgagcagaatgtcatcgaacaacctggctcttccttggcatccaaccggatattgactcttcctcagcgcacaatacgaggcaagaataaacattgttggtccaccagtaaatcaacgcgccggtcaagagtttctgcccttaatatcgtccgaagtcaaaggggaccaacaagaatgtgccgcaacatatatgatcctttgttgtgctttaaactttttttcactgacgaaatcatatctgaaatcgtcaagtggaccaacgcagagatttccctgaagagaagggagtccatgaccagcgctacatttcgggataccaatgaagacgagatatacgcattcttcggaatattggtgatgaccgcagttcgcaaggacaaccacatgtctaccgatgatttgttcgatagaagcctctctatggtctatgtaagtgtgatgtcccgggataggtttgattttcttatacgatgccttagaatggacgataaatcaatacggcccacgttgcgagaaaacgatgtatttaccccagtgcggaaaatctgggacctcttcatccaccaatgcatacaaaactatacgcccggggcacatcttactattgacgaacaactcctcggctttagggggcggtgtccatttagagtctacattccaaataagcctagtaaatacgggattaaaatactcatgatgtgcgattccgggaccaaatatatgataaacggaatgccatatttgggtagagggacccagacaaacggagtgccactgggggaatactatgtgaaagagttgtcaaaaccagtacacggttcttgtcgcaacataacctgcgataactggttcacatccatcccccttgctaagaacctgcttcaggaaccttacaaattgacgatagttggaactgtcaggtcaaataagagggagataccggaagttttgaagaatagtcgatctcgaccagtcggtacctcaatgttttgcttcgacggacccctgacactggtttcatacaagcctaagcccgccaagatggtgtatctcctgtccagctgtgacgaagacgcctcaatcaacgaatcaaccggcaaaccccaaatggtgatgtattataatcaaacgaagggaggcgtcgatactttggatcagatgtgcagtgttatgacatgcagtcgcaaaactaatcggtggcccatggctctcctctatgggatgattaacattgcttgtataaactcctttatcatctactcccataacgtatctagtaagggtgagaaagtacagagccggaaaaagttcatgaggaatttgtacatgagtctgacgagttccttcatgaggaaacggctcgaagctccaactcttaaaagatatctcagagacaacatttccaatatccttcctaaagaagtccccgggacgagtgatgatagcactgaagaacccgtgatgaaaaagcgcacgtactgcacctattgtccttccaagattcggcggaaggctaatgcatcttgtaaaaaatgtaaaaaggttatttgccgagaacacaacattgatatgtgccagtcatgcttttgacaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctgacgcgtgcatgcgtcaattttacgcagactatctttctagggttaagcgctagcagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagactagt

SEQ ID NO: 5 - DNA, 2735 bp, Artificial Sequence
  rAAV-PB-dTK construct
accggtactgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcttaagggttaaccctagaaagataatcatattgtgacgtacgttaaagataatcatgcgtaaaattgacgcatgaatccctagggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattgcgaattcgccgccaccatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgacaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctgacgcgtgcatgcgtcaattttacgcagactatctttctagggttaagcgctagcagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtagcgagcgagcgcgcagactagt